(19) United States
(12) Patent Application Publication
(10) Pub. No.: US 2005/0059156 A1
(43) Pub. Date: Mar. 17, 2005

(54) Method for detecting contaminants in pharmaceutical products

(75) Inventors: Vincenzo Rizzo, Arese (IT); Mario Arangio, Milano (IT); Giuseppe Razzano, Magenta (IT); Maristella Colombo, Milano (IT); Federico Riccardi Sirtori, Milano (IT)

Correspondence Address: SCULLY SCOTT MURPHY & PRESSER, PC, 400 GARDEN CITY PLAZA, GARDEN CITY, NY, 11530, US

(73) Assignee: Pharmacia Italia S.p.A., Milan (IT)

(21) Appl. No.: 10/912,318

(22) Filed: Aug. 5, 2004

(30) Foreign Application Priority Data
Aug. 6, 2003 (EP) 03102444.1

(51) Int. Cl. G01N 33/00; B01D 59/44

(52) U.S. Cl. 436/94

(57) ABSTRACT

The present invention relates to a quantitative, highly sensitive analytical procedure capable to control at part per billion (ppb) detection level the possible presence of undesired chemical contaminants, especially β-lactam antibiotics, in anthracycline products.

### CROSS REFERENCE TO RELATED APPLICATIONS

This application is based upon and claims the benefit of priority from European Patent Application No. 03102444.1 filed on Aug. 6, 2003, the entire contents which is expressly incorporated herein by its reference.

### BACKGROUND OF THE INVENTION

Undesired organic chemical contamination of pharmaceutical products is a significant problem in the pharmaceutical industry.

The qualitative and quantitative detection of organic chemical substances that may contaminate pharmaceutical products is an issue that has focused interest on the development of appropriate analytical techniques.

Residues of chemical contaminants, especially antibiotics, are commonly investigated in milk. Typical tests include immunoaffinity, microbial or receptor based screening which, in most cases, cannot identify specific chemical products.

Many different analytical methods using liquid chromatography (LC) with mass spectrometry (MS) detection have been also reported in literature for detection of β-lactam residues in milk with application of different mass analyzers (see, e.g., D. N. Heller and M. A. Ngoh “Electrospray Ionization and Tandem Ion Trap Mass Spectrometry for the Confirmation of seven β-lactam antibiotics” Rapid Commun. Mass Spectrom. 12:2031-2040 (1998); E. Daeseleire, H. De Ruyck and R. Van Renterghen “Confirmatory assay for the simultaneous detection of penicillins and cephalosporins in milk using liquid chromatography/tandem mass spectrometry” Rapid Commun. Mass Spectrom. 14:1404-1409 (2000); S. Riediker, J. Diserens and R. H. Stadler “Analysis of β-lactam antibiotics in incurred raw milk by rapid test methods and liquid chromatography coupled with Electrospray Ionization Tandem Mass Spectrometry” J. Agric. Food Chem. 49:4171-4176 (2001); and F. Bruno, R. Curini, A. Di Corcia, M. Nazzari and R. Sampieri “Solid-Phase Extraction followed by liquid chromatography-mass spectrometry for trace determination of β-lactam antibiotics in bovine milk” J. Agric. Food Chem. 49:3463-3470 (2001)). All of these methods use electrospray (ES) as ion source; in some cases tandem mass spectrometry (MS/MS) is also applied. Limits of detection below 10 ppb are achieved for several β-lactam compounds in milk using multiple analyser configuration, i.e. triple quadrupole instruments (see E. Daeseleire et al. and S. Riediker above). The same sensitivity is obtained by utilizing an ion trap mass analyser (see D. N. Heller et al. above). This latter analyzer provides full scan MS/MS performance at full instrument sensitivity, in contrast to triple quadrupole MS/MS, which requires a selection of only a few ions to maximize sensitivity. The applicability of the above reported tests to other substrates, such as pharmaceutical products, particularly to anthracycline products, is considered a real challenge due to the presence of an almost a million fold higher concentration of anthracycline product versus the contaminants.

A method has been developed by the present inventors as quantitative analytical procedure for detection of any class of organic chemical contaminants, particularly β-lactam compounds, in anthracycline products, following a LC-MS/MS approach similar to the above known procedures combining liquid chromatographic separation with mass spectrometric determination.

Former experiments were performed using Selected Ion Monitoring detection mode; for each compound the [M+H]⁺ ion was monitored. Later on, a MS/MS method was developed to increase selectivity and improve sensitivity. This technique is called Selected Reaction Monitoring (SRM).

Selected reaction monitoring is a two-stage technique in which parent ion and daughter ion pairs are monitored. In the first stage of mass analysis, the ions formed in the ion source are stored in the mass analyzer (ion trap). Ions of a given mass-to-charge ratio (parent ions) are selected and all other ions are ejected from the mass analyzer. Then, the parent ions are excited so that they collide with background gas that is present in the mass analyzer.

The collisions of the parent ions cause them to fragment to produce one or more daughter ions. In the second stage of mass analysis, the daughter ions are stored in the ion trap. Ions of one or more mass-to-charge ratios are selected and all other ions are ejected from the mass analyzer. Then, the selected product ions are scanned out of the mass analyzer and monitored.

For each compound the [M+H]⁺ ion was determined and dissociation with radio frequencies of the molecular ion was induced. The MS system was tuned to obtain the optimal conditions for generating the daughter ions.

When the anthracycline product was present as the active ingredient in a final dosage form comprising a large amount of an inert excipient, solid-phase extraction procedure (SPE) has been carried out in order to isolate and concentrate the possible contaminants and to wash away the interfering excipient, before starting liquid chromatographic fractionation.

The above method, however, does not allow achieving a satisfying separation among anthracycline products and organic chemical contaminants, especially all β-lactam antibiotics, because the remaining excess of the anthracycline product present in the column effluent prevents the β-lactams determination, especially in case of the most hydrophobic ones, even if a good chromatographic separation is obtained.

Accordingly, there is a need to develop a method, which improves assay performance characteristics such as sensitivity, thus enabling these organic chemical contaminants, especially β-lactam antibiotics, to be detected in anthracycline products in a manner, which is both precise and highly sensitive.

The present invention fulfils such a need by introducing a further preparation step comprising precipitation of the anthracycline through complexation with DNA (see Wang A. H.-J. et al. Biochemistry 26:1152-1163 (1987)) immediately before LC so that the interfering anthracycline product is almost completely removed from the supernatant. The introduction of this new step increases the sensitivity of the above-identified method, by improving the limit of detection (LOD) of the chemical organic contaminants more than thirty-fold over the previous method.

### SUMMARY OF THE INVENTION

It is therefore a first object of the present invention a quantitative analytical method for assessing the presence of an organic chemical contaminant in an anthracycline product, which comprises the steps of:

(a) fractionating said contaminant with an appropriate liquid chromatography (LC) apparatus;

(b) detecting and determining said contaminant in the LC column effluent with a highly sensitive mass spectrometry (MS) apparatus;

characterized in that the sensitivity of the method is increased by selectively precipitating interfering anthacycline with DNA solution to form the insoluble anthracycline-DNA complex that is removed by centrifugation prior to step (a).

The limit of detection of organic chemical contaminants, particularly β-lactam antibiotics, achieved by the method according to the invention is below 6 ppb.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Standard Addition Method of β-lactam antibiotics (Method A): Amoxicillin. Equation data: r²=0.9937; LOD=242 ppb

FIG. 2. Standard Addition Method of β-lactam antibiotics (Method A): Cefazolin. Equation data: r²=0.9983; LOD=142 ppb

FIG. 3. Standard Addition Method of β-lactam antibiotics (Method B): Amoxicillin. Equation data: r²=0.9965; LOD=2.7 ppb

FIG. 4. Standard Addition Method of β-lactam antibiotics (Method B): Cefazolin. Equation data: r²=0.9935; LOD=4.1 ppb

FIG. 5. Limit Test of amoxicillin at 6 ppb in reconstituted adriblastin lyophile (Method B): A. Sample solution B. Sample solution spiked with 6 ppb of amoxicillin.

FIG. 6. Limit Test of cefazolin at 6 ppb in reconstituted adriblastin lyophile (Method B): A. Sample solution. B. Sample solution spiked with 6 ppb of cefazolin.

FIG. 7. Limit Test of ceftizoxime at 6 ppb in reconstituted adriblastin lyophile (Method B): A. Sample solution. B. Sample solution spiked with 6 ppb of ceftizoxime.

FIG. 8. Limit Test of ampicillin at 6 ppb in reconstituted adriblastin lyophile (Method B): A. Sample solution. B. Sample solution spiked with 6 ppb of ampicillin.

FIG. 9. Limit Test of penicillin V at 6 ppb in reconstituted adriblastin lyophile (Method B): A. Sample solution. B. Sample solution spiked with 6 ppb of penicillin V.

### DETAILED DESCRIPTION OF THE INVENTION

Preferably, the LC apparatus according to the present invention is a reversed-phase high performance liquid chromatography (HPLC) apparatus and the highly sensitive mass spectrometry (MS) apparatus is a tandem mass spectrometry (MS/MS) apparatus, such as, e.g., triple quadrupole, ion trap, QTof, double focusing magnetic sector, equipped with an electrospray (ES) ionisation device.

The anthracycline product potentially containing an organic chemical contaminant may be either as active product ingredient or included in a finished dosage form. The anthracycline product potentially containing an organic chemical contaminant may be of either a liquid or solid form. In the case of solid, the anthracycline product is first reconstituted, i.e. made into a liquid form.

As already stated above, when the anthracycline product is present as the active ingredient in a final dosage form which comprises a large amount of an inert excipient such as, for example, lactose in the lyophilised form of doxorubicin hydrochloride (commercially available as ADRIAMYCIN® or ADRIBLASTIN®), the hydrophilic excipient is preliminarily washed away with water by using solid-phase extraction (SPE) cartridges.

It is therefore a further aspect of the present invention a quantitative analytical method as described above which also comprises, if required, a preliminary SPE of the organic chemical contaminants for removing the interference caused by the excipient.

The term “detecting” means qualitatively analysing for the presence or absence of a contaminant.

The term “determining” means quantitatively analysing for the amount of a contaminant.

The term “anthracycline product” means an anticancer agent consisting of 3 moieties: a pigmented aglycone, an amino sugar, and a lateral chain. Examples include doxorubicin, epirubicin, idarubicin, and daunorubicin.

The term “organic chemical contaminant” includes one or more synthetic or natural carbon-containing molecules that, either by accident or by mischievous purpose, have become intimately mixed with the pharmaceutical product, particularly β-lactam antibiotics.

The term “limit of detection,” means the lowest amount of organic chemical contaminating a sample that can be detected, but not necessarily determined. The limit of detection is expressed as the concentration of organic chemical contaminant (part per billion, ng/g) in the sample.

The term “β-lactam antibiotic” means a class of broad spectrum antibiotics that are structurally and pharmacologically related to the penicillins including ampicillin, amoxicillin, azlocillin, bacampicillin, cefixime, carbenicillin, methicillin, cloxacillin, 6-APA, piperacillin, pivmecillinam, penicillin V, monolactam, aztreonam, mecillinam, imipenem, meropenem; and cephalosporins including cefoperazone, latamoxef, cephapirin, cefazolin, cefaclor, ceftibuten, ceftizoxime, cefotetan, cefuroxime, cefprozil, ceftazidime, cephaloglycine, cephaloridine, nitrocephine, cefatoxime, ceftiofur, cephapyrine, cefepime, cefpirome, cefadroxil, cefamandole, cefoxitin, cefpodoxime, ceftriaxone, cephalexin, cephazoline, cephradine and 7-ACA.

In a particular aspect, the method according to the present invention combines selective precipitation of the complex DNA-anthracycline, reversed-phase liquid chromatography, electrospray (ES) ionization and tandem ion trap mass spectrometry (MS/MS) and, if required, preliminary solid-phase extraction (SPE). The ion trap analyzer can selectively store and accumulate the target ion and, after a collision-induced dissociation (CID), provide full scan MS/MS performance at top instrument sensitivity. This process produces fragmentation patterns that permit clear identification of target organic chemical contaminants and their determination using a specific MS/MS transition.

More in particular, the method according to the present invention has been developed for achieving a LOD below 6 ppb for β-lactam antibiotics, such as, e.g., amoxicillin, cefazolin, ceftizoxime, ampicillin and penicillin V, in lyophilised anthracycline products, typically doxorubicin hydrochloride. Values refer to a weight by weight basis for the solid product.

If present, a hydrophilic excipient (e.g. lactose) is washed away with water by using SPE cartridges. The active principle, e.g. doxorubicin hydrochloride, is selectively cleaned up by adding a DNA solution to form the insoluble anthracycline-DNA complex that is removed by centrifugation. Supernatant solution is then directly analyzed.

Good separation is obtained between the different β-lactam antibiotics and the residual active ingredient (originally present in overwhelming quantity).

For each β-lactam antibiotic a detection limit below 6 ppb is achieved, with satisfactory linearity, as obtained by standard addition technique, in the range 3-18 ppb.

The chosen criterion for the limit test [the quotient between the area of the sample solution and the spiked sample solution (at 6 ppb) must not exceed 0.5] is always fulfilled.

The following example shows the detection and determination of β-lactam antibiotics, in doxorubicin hydrochloride (ADRIBLASTIN®) as a representative example of anthracycline products according to the present invention, without, however, limiting its scope.

Those skilled in the art will appreciate that the method taught in the example is also applicable to the detection and determination of organic chemical contaminants different from β-lactam antibiotics, not only in doxorubicin hydrochloride, but also in other anthracycline products.

### EXAMPLE 1

### Determination of Antibiotics Containing A β-Lactam Ring in Doxorubicin Hydrochloride

Comparison of Detection Limits as Obtained with Standard Reference Procedure (Method A) and with the Method of Present Invention (Method B).

Materials and Instrumentations

| Apparatus | Model | Source |
|---|---|---|
| Mass Spectrometer | LCQ Deca | Finnigan |
| HPLC chromatograph | P4000 | Thermo Separation |
| | | Products |
| Autosampler | LC-PAL | CTC Analytics |
| Analytical balance | RC 210 D | Sartorius |
| Technical balance | B303 | Mettler Toledo |
| pH-meter | E603 | Metrohm |
| Water purifier | Milli-Q Gradient A10 | Millipore |
| Ultrasonic bath | 220 | Bransonic |

| Chemicals & Consumables | Source |
|---|---|
| Acetonitrile | Fluka |
| Ammonium Acetate | Carlo Erba |
| Formic Acid | Carlo Erba |
| Oasis ® HLB cartridge 3 ml 60 mg | Waters |
| DNA free acid from herring sperm (D3159) | Sigma |
| Samples & Standards | Source |
| Adriblastin, batch 2V309 | Pharmacia |
| 300 mg (50 mg doxorubicin) | |
| Amoxicillin trihydrate, 500 mg 200005WPP | Pharmacia |
| Cefazolin, 500 mg 200013WOP | Pharmacia |
| Ceftizoxime sodium, 500 mg 200014WPP | Pharmacia |
| Ampicillin trihydrate, 500 mg 200006WPP | Pharmacia |
| Penicillin V, 500 mg 200073WPP | Pharmacia |

Preparation of the Sample and Standard Solutions
Solution A (β-Lactam Standard 50 μg/mL)

2.5 mg of each β-lactam were accurately weighed and transferred in a 50 mL volumetric flask. About 5 mL of acetonitrile were added. The suspension was stirred until total dissolution and brought to volume with 5 mM ammonium acetate buffer, pH 5.5.

Solution B (β-Lactam Standard 100 ng/mL)

A volume of 100 μL of solution A was transferred into a 50 mL volumetric flask and brought to volume with 5 mM ammonium acetate buffer, pH 5.5.

DNA Solution (50 mg/mL)

500 mg of DNA free acid from herring sperm was accurately weighed and transferred in a 50 mL becker. 10 mL of buffer 5 mM ammonium acetate buffer, pH 5.5 were added and the suspension was stirred one night at room temperature to favor DNA dissolution.

About 5 mL of acetonitrile were added. The suspension was stirred until total dissolution and brought to volume with 5 mM ammonium acetate buffer, pH 5.5.

Sample Preparation

Method A

A vial of Adriblastin lyophile (300 mg solid mass, containing 50 mg of doxorubicin) was dissolved into 12.5 mL of 5 mM ammonium acetate buffer, pH 5.5. Solutions, for standard addition method, were fortified at this point by aliquoting the 100 ng/mL mixed standard as shown in Table 1.

The final concentration of doxorubicin solutions is 2 mg/mL.

TABLE 1
Composition of the solutions for standard
addition quantitation (Method A)

| | | | | | | Concentration |
|---|---|---|---|---|---|---|
| | | | | | Concen- | of β-lactam |
| | Adriblastin | Buff- | β- | Added | tration | Added β- |
| | solution | er pH | lactam | β- | of β- | lactam/weight |
| Solu- | 4 mg/ml | 5.5 | solution | lactam | lactam | of lyophile |
| tion | [μL] | [μL] | B [μL] | [ng] | [ng/mL] | [ng/g] or [ppb] |
| L1 | 500 | 470 | 30 | 3 | 3 | 250 |
| L2 | 500 | 440 | 60 | 6 | 6 | 500 |
| L3 | 500 | 410 | 90 | 9 | 9 | 750 |
| L4 | 500 | 320 | 180 | 18 | 18 | 1500 |
| L0 | 500 | 500 | 0 | 0 | 0 | 0 |

Method B

A vial of Adriblastin lyophile (300 mg solid mass, containing 50 mg of doxorubicin) was dissolved into 3 mL of 5 mM ammonium acetate buffer, pH 5.5. Solutions, for standard addition method, were fortified as shown in Table 2.

TABLE 2
Composition of the solutions for standard
addition quantitation (Method B)

| | | | | Concentration |
|---|---|---|---|---|
| | | | | Added β- |
| | Reconstituted | β-lactam | Added | lactam/weight |
| | adriblastin | solution | β-lactam | of lyophile (300 mg) |
| Solution | lyophile [mL] | B [μL] | [ng] | [ng/g] or [ppb] |
| L1 | 3.000 | 9 | 0.9 | 3 |
| L2 | 3.000 | 18 | 1.8 | 6 |
| L3 | 3.000 | 36 | 3.6 | 12 |
| L4 | 3.000 | 54 | 5.4 | 18 |
| L0 | 3.000 | 0 | 0.0 | 0 |

The Adriblastin solutions, spiked with β-lactam, were loaded on Waters Oasis® cartridges (HLB 3 mL, 60 mg) which were completely drained of liquid under vacuum and washed with 1 mL of ammonium acetate 5 mM pH 5.5 buffer.

Analytes were eluted using 2 mL of methanol (2×1 mL). A vacuum was applied to completely drain the SPE cartridge.

The extract was evaporated to dryness using a Savant SpeedVac Concentrator at a temperature of 25° C. for 90 minutes. The residue was dissolved into 400 μL of methanol/5 mM ammonium acetate buffer pH 5.5 50/50. 150 μl of DNA solution were added under stirring; a red solid formed.

The mixture was transferred in eppendorf tubes and centrifuged for 3 minutes at 12000 rpm. A volume of 60 μL of supernatant was injected on the HPLC-MS system.

HPLC-MS Conditions

Mass spectra analyses were conducted on a Finnigan LCQ Deca ion trap mass spectrometer equipped with an Electrospray (ESI) ion source. The mass spectrometer is directly connected to a Spectra System P4000 HPLC pump (Thermo Separation Products), equipped with an Lc PAL Autosampler (CTC Analytics). For sample injection, a Vici injector, equipped with a 20 μL loop is used. The system is controlled by the version 1.2 of the Excalibur Software.

| | |
|---|---|
| Column: | Alltech Solvent Miser C18 5 mm (250 × 2.1 mm) |
| | equipped with a guard cartridge Phenomenex C8 |
| | (4 mm L × 3 mm ID). |
| Mobile phase A: | water acidified with 0.1% formic acid |
| Mobile phase B: | acetonitrile acidified with 0.1% formic acid |
| Flow rate: | 0.4 mL/min |

| | | |
|---|---|---|
| Elution: | Time (min) | % Mobile Phase A |
| | 0 | 90 |
| | 30 | 45 |
| | 31 | 0 |
| | 35 | 0 |
| | 36 | 90 |
| | 46 | 90 |

Electrospray with positive ions detection was used as ionization mode.

Tuning of the LC/MS instrument was performed by infusing solutions, containing 100 ng/mL of the compounds in a 0.1% solution of formic acid. The syringe pump was operated at a flow rate of 10-20 μL/min. For each compound the ESI source parameters were optimised. β-lactam antibiotics were divided in two sets, that were analyzed individually. The instrument parameters changed during the analyses to optimise the response for each compound. 60 μL of sample solution were injected in a 20 μL loop.

Scan and MS/MS parameters are shown in Table 3.

TABLE 3
Mass scan parameters for each β-lactam

| | | | Daughter ions | | MSⁿ | |
|---|---|---|---|---|---|---|
| | β- | | (m/z) used for | Collision | Scan | Ret. Time |
| | lactam | Parent | integration | Energy | Range | window |
| Compound | set | ion (m/z) | (SRM) | (%)^{a} | (m/z) | (min) |
| Amoxicillin | A | 366 | 349 | 24 | 348-350 | 0-7.5 |
| Cefazolin | A | 455 | 323 | 20 | 322-324 | 7.5-14 |
| Ceftizoxime | B | 383 | 241 | 25 | 105-380 | 0-10.30 |
| Ampicillin | B | 350 | 160 | 30 | 159.5-160.5 | 10.30-18.30 |
| Penicillin V | A | 351 | 160 | 35 | 159.5-160.5 | 19-46 |

^{a}arbitrary units

Results
Standard Addition Method β-Lactam Antibiotics

In order to evaluate linearity and limit of detection of the analytical procedure the standard addition method was performed on Adriblastin lyophile.

From the mixed β-lactam antibiotics stock solution (100 ng/mL) four Adriblastin lyophile were spiked in the concentration range 3-18 ppb (250-1500 ppb for Method A) and treated as shown in the experimental part.

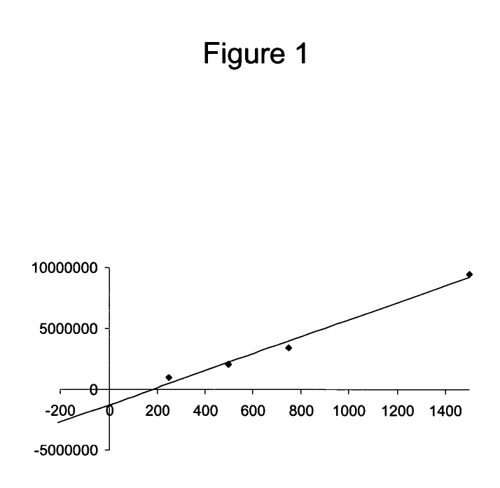

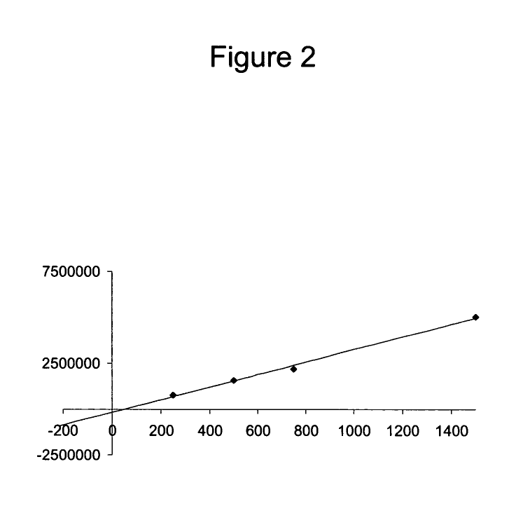

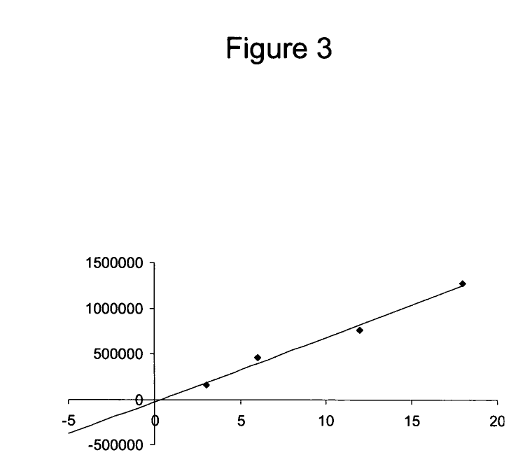

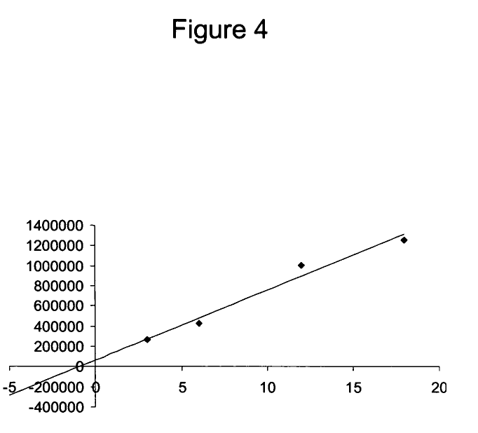

Details on the preparation of the sample solutions (L0, . . . , L4) are given in Table 1. (Method A) and in Table 2 (Method B). For each of the β-lactam antibiotics a limit of detection below 5 ppb is achieved, applying the method including DNA precipitation (Method B), with satisfactory linearity. An estimate of the limit of detection (3σ/b) is obtained from the standard deviation of the data (σ) and the method sensitivity (b). The results for both methods are shown in Table 4 and in FIGS. 1-4.

TABLE 4
Comparison of Linearity and LOD between Method A and Method B

| | | Amoxicillin | Cefazolin | Ceftizoxime | Ampicillin | Penicillin V |
|---|---|---|---|---|---|---|
| Method A | R² | 0.9937 | 0.9983 | 0.9984 | 0.9899 | 0.9943 |
| | LOD (ppb) | 242 | 142 | 133 | 283 | 291 |
| Method B | R² | 0.9965 | 0.9935 | 0.9981 | 0.9973 | 0.9964 |
| | LOD (ppb) | 2.7 | 4.1 | 1.8 | 2.1 | 3.7 |

Reproducibility

Five lyophiles, spiked at 500 ppb for Method A and at 6 ppb for Method B, have been treated and analysed for an assessment of method reproducibility. Results are collected in Table 5 and show that relative standard deviation (RSD) is maintained below 25% for each β-lactam antibiotic in either procedure.

TABLE 5
Observed areas and reproducibility for
β-lactam antibiotics in Adriblastin

| | | | | Ampi- | Penicillin |
|---|---|---|---|---|---|
| | Amoxicillin | Cefazolin | Ceftizoxime | cillin | V |
| RSD | 6.6% | 9.7% | 9.9% | 12.6% | 21.3% |
| Method A | | | | | |
| RSD | 11.7% | 16.4% | 10.1% | 17.6% | 24.4% |
| Method B | | | | | |
| DNA | | | | | |
| treatment | | | | | |

Limit of Detection

The acceptance criterion for limit of detection is a signal-to-noise not less than 3. LOD was calculated by Excalibur 1.2 Software. Signal to noise is given by the ratio of the signal height (S) to the noise height (N). The signal height is the baseline corrected peak height. The noise height is the residual difference, r (max)-r (min), from a linear regression analysis of the baseline noise. The criterion was fulfilled.

Limit Test with DNA Treatment

The chosen criterion for limit test is that the quotient between the area of the sample solution and the fortified sample solution (spiked at 6 ppb) must not exceed 0.5.

Limit test analyses were performed using DNA treatment that permits to achieve the limit of detection requested. A prerequisite for this method is the linearity between the concentration of the analytes and the corresponding peak area. That was tested in the standard addition method (see Table 4).

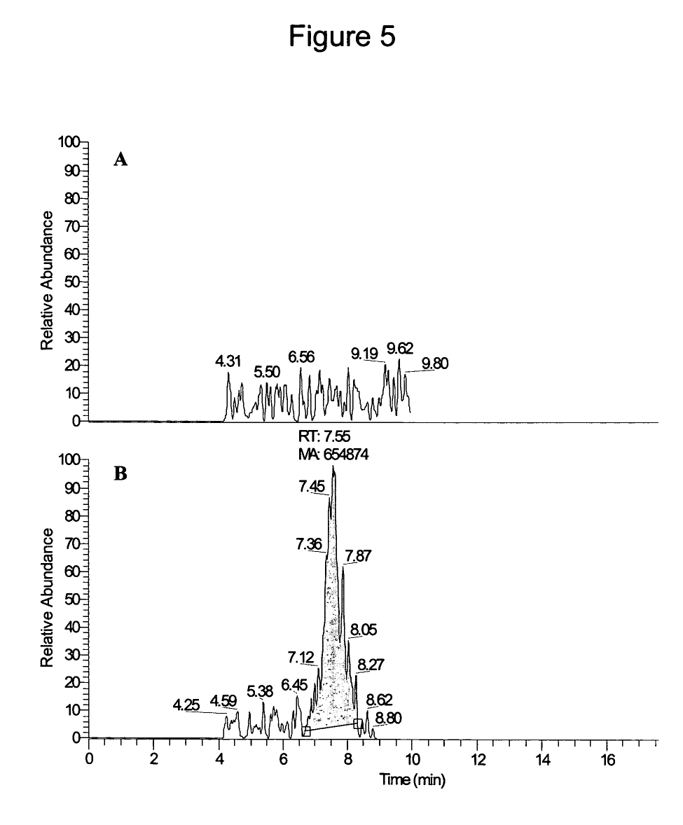

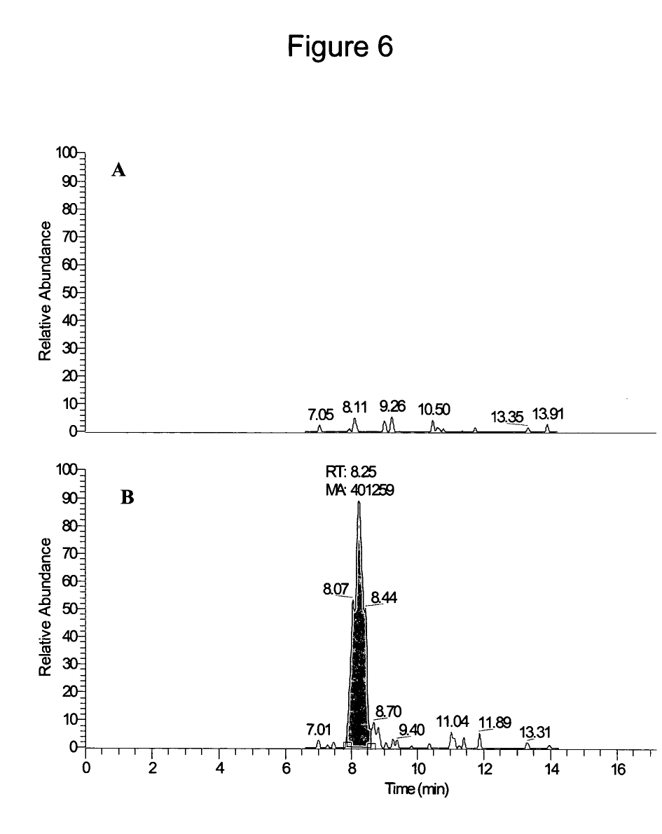

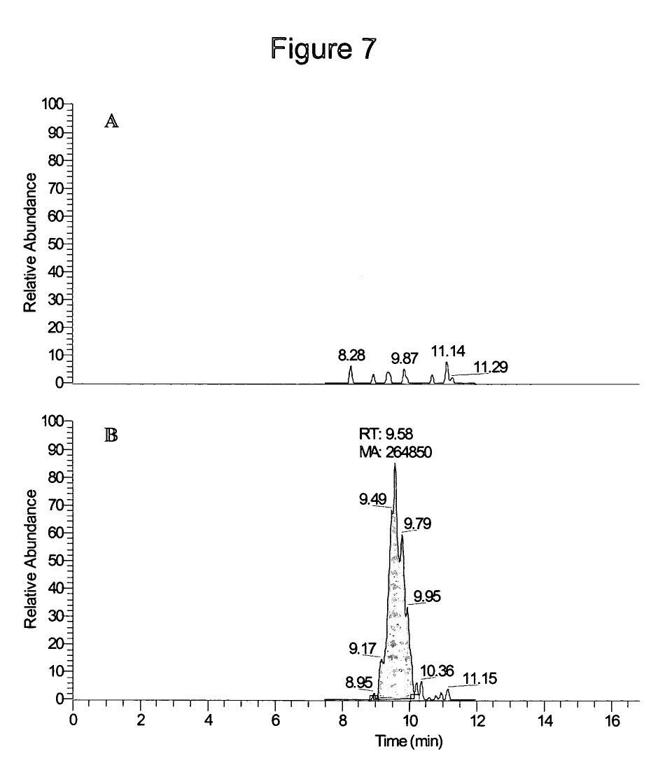

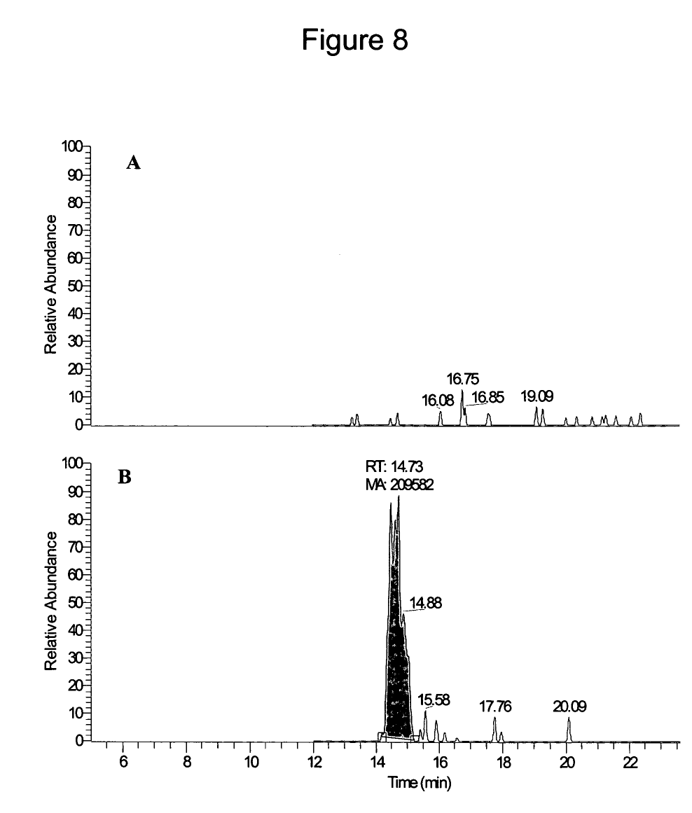

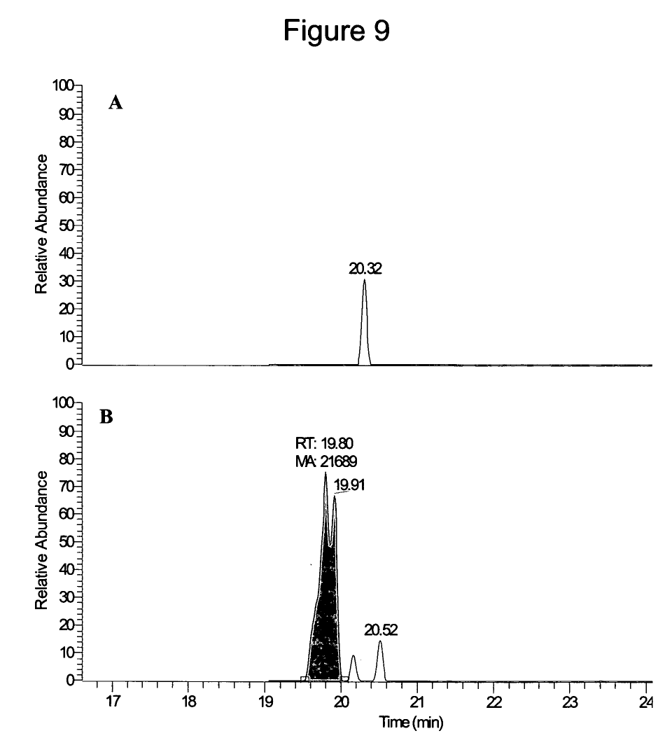

For all β-lactam antibiotics the quotient S₀/S₆ was below the limit of 0.5. Chromatograms are shown on FIGS. 5-9.

TABLE 6
Results of Limit Test

| | Amoxi- | | | Ampi- | Penicillin |
|---|---|---|---|---|---|
| | cillin | Cefazolin | Ceftizoxime | cillin | V |
| Spiked 0 ppb | 0 | 0 | 0 | 0 | 0 |
| S₀ (area units) | | | | | |
| Spiked 6 ppb | 654874 | 401259 | 264850 | 209582 | 21689 |
| S₆ (area units) | | | | | |
| Quotient 1 | 0 | 0 | 0 | 0 | 0 |
| S₀/S₆ | | | | | |

1. A quantitative analytical method for assessing the presence of an organic chemical contaminant in an anthracycline product, which comprises the steps of:
(a) fractionating said contaminant with an appropriate liquid chromatography (LC) apparatus;
(b) detecting and determining said contaminant in the LC column effluent with a highly sensitive mass spectrometry (MS) apparatus;
characterized in that the sensitivity of the method is increased by selectively precipitating interfering anthacycline with DNA solution to form the insoluble anthracycline-DNA complex that is removed by centrifugation prior to step (a).

2. A method according to claim 1, wherein the limit of detection of the organic chemical contaminant is below 6 ppb.

3. A method according to claim 1 or 2, which also comprises if required, a preliminary solid phase extraction (SPE) of the organic chemical contaminant for removing the interference caused by the excipient.

4. A method according to anyone of the preceding claims, wherein the chemical contaminant is a β-lactam antibiotic.

5. A method according to anyone of the preceding claims, wherein the anthracycline product is doxorubicin, epirubicin, idarubicin, or daunorubicin.

6. A method according to claim 5, wherein the anthracycline product is doxorubicin.

7. A method according to claim 4, wherein the β-lactam antibiotic is ampicillin, amoxicillin, azlocillin, bacampicillin, cefixime, carbenicillin, methicillin, cloxacillin, 6-APA, piperacillin, pivmecillinam, monolactam, aztreonam, mecillinam, imipenem, meropenem, cefoperazone, latamoxef, cephapirin, cefazolin, cefaclor, ceftibuten, ceftizoxime, cefotetan, cefuroxime, cefprozil, ceftazidime, cephaloglycine, cephaloridine, nitrocephine, cefatoxime, ceftiofur, cephapyrine, cefepime, cefpirome, cefadroxil, cefamandole, cefoxitin, cefpodoxime, ceftriaxone, cephalexin, cephazoline, cephradine or 7-ACA.

8. A method according to claim 7, wherein the β-lactam antibiotic is ampicillin, amoxicillin, cefazolin, ceftizoxime, or penicillin V.
